(19) United States
(12) Patent Application Publication
(10) Pub. No.: US 2026/0337021 A1
(43) Pub. Date: Oct. 29, 2026

(54) ATR REFORMING

(71) Applicant: Topsoe A/S, Kgs. Lyngby (DK)

(72) Inventors: Arunabh SAHAI, Haryana (IN); Kim AASBERG-PETERSEN, Allerød (DK); Thomas Sandahl CHRISTENSEN, Kgs. Lyngby (DK); Steffen Spangsberg CHRISTENSEN, Køge (DK)

(73) Assignee: Topsoe A/S, Kgs. Lyngby (DK)

(21) Appl. No.: 19/111,538

(22) PCT Filed: Sep. 15, 2023
(86) PCT No.: PCT/EP2023/075456

§ 371 Date: Mar. 13, 2025

(30) Foreign Application Priority Data
Sep. 16, 2022 (IN) 202211053160
Feb. 10, 2023 (EP) 23156113.5

(51) Int. Cl. C01B 3/382; C01B 3/50; C01C 1/04; C07C 29/152; C10K 3/02

(52) U.S. Cl. CPC C01B 3/382; C01B 3/50; C01C 1/0405; C07C 29/152; C10K 3/026; C01B 2203/0233; C01B 2203/0244; C01B 2203/061; C01B 2203/068; C01B 2203/085; C01B 2203/1241; C01B 2203/127

(57) ABSTRACT

The present invention relates to a synthesis gas stage in which an electrical heating unit is located between a prereformer section and an ATR section. The first electrical heating unit is arranged to heat the prereformed stream to at least 400 °C prior to it being fed to the ATR section. The invention also relates to a process for producing a first syngas stream, a hydrogen plant, which can be used in hydrogen, ammonia, methanol and synthetic fuel production plants and a method for reducing CO₂ emissions in a hydrogen plant.

### TECHNICAL FIELD

The present invention relates to a synthesis gas stage in which an electrical heating unit is located between a prereformer section and a reforming section. The first electrical heating unit is arranged to heat the prereformed stream to at least 400 °C prior to it being fed to the reforming section. In this manner, one or more fired heaters can be avoided. The invention also relates to a process for producing a first syngas stream, and to a production plant such as a hydrogen plant, an ammonia plant, a methanol plant, and a synthetic fuel production plant comprising the described synthesis gas stage as well as to a method for reducing CO₂ emissions in said plants.

### BACKGROUND

Significant efforts have been put into optimizing production for hydrogen plants, with the objective to improve overall energy efficiency and reduce capital cost. The need for more cost-efficient hydrogen production has spurred the development of technology and catalysts for large-scale hydrogen production units, in order to benefit from economy of scale. Efforts have also been made to reduce greenhouse gas outputted from such plants.

It would be advantageous to reduce—among other things—the CO₂ emissions from hydrogen plants.

WO2022038089 describes a plant for producing a hydrogen-rich stream from a hydrocarbon feed.

### SUMMARY

It is an object of the present invention to reduce consumption of hydrocarbon feed and fuel in a hydrogen plant and/or process, thereby increasing energy efficiency. At the same time, it is an object to reduce CO₂ emissions in a hydrogen plant and/or process. The carbon footprint of the plant can thereby be significantly reduced.

These and other objects are addressed by the present invention.

Accordingly, in a first aspect, a synthesis gas stage is provided, said stage comprising:
a first hydrocarbon feed,
a prereformer section, arranged to receive the first hydrocarbon feed and provide a prereformed stream,
a reforming section, arranged to receive the prereformed stream and provide a first syngas stream,
   wherein said synthesis gas stage comprises a first electrical heating unit located between said prereformer section and said reforming section, said first electrical heating unit being arranged to heat the prereformed stream to at least 400 °C, preferably at least 450 °C, prior to being fed to the reforming section.

A process for producing a first syngas stream in the synthesis gas stage is also provided, said process comprising the steps of:
a. feeding the first hydrocarbon feed to the prereformer section, and prereforming the first hydrocarbon feed to provide a prereformed stream,
b. feeding the prereformed stream to the first electrical heating unit, and heating the prereformed stream,
c. feeding the heated, prereformed stream to the reforming section, and subjecting it to reforming to provide a first syngas stream.

Further provided is a hydrogen plant, an ammonia plant, a methanol plant and a synthetic fuel production plant comprising the synthesis gas stage described herein.

Including an electrical heater enables a smooth plant start-up. Electrical heater with a renewable power supply increases the overall carbon efficiency of the plant.

Further details of the invention are set out in the following description, following figures, aspects and the dependent claims.

### LEGENDS

The technology is illustrated by means of the following schematic illustrations, in which:

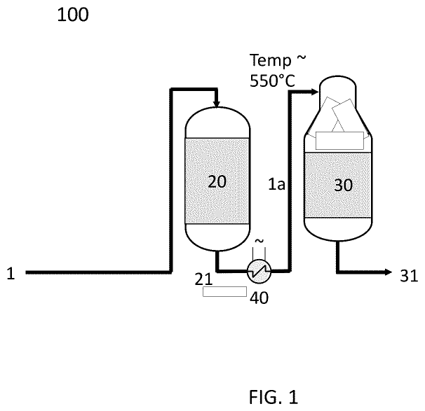

FIG. 1 shows a simple layout of one aspect of the system of the invention.

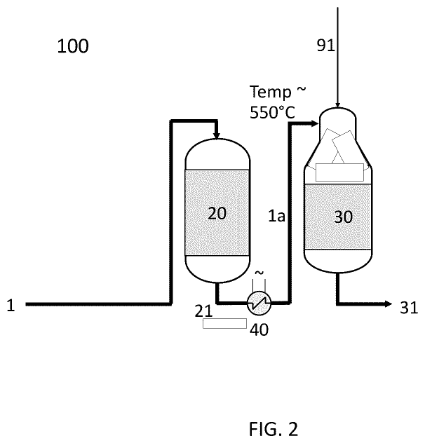

FIG. 2 shows a more developed layout of the system of the invention.

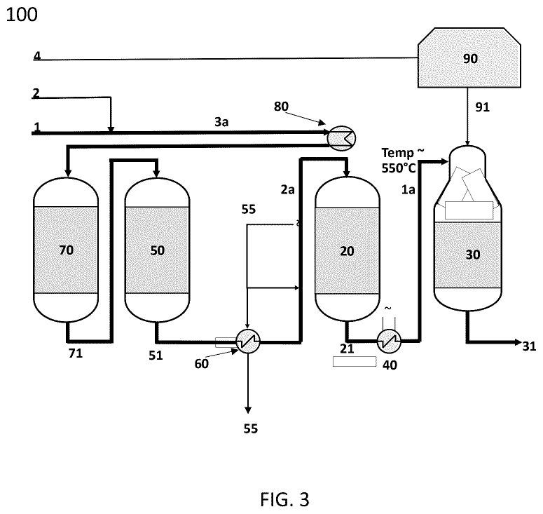

FIG. 3 shows a further developed layout of the system of the invention.

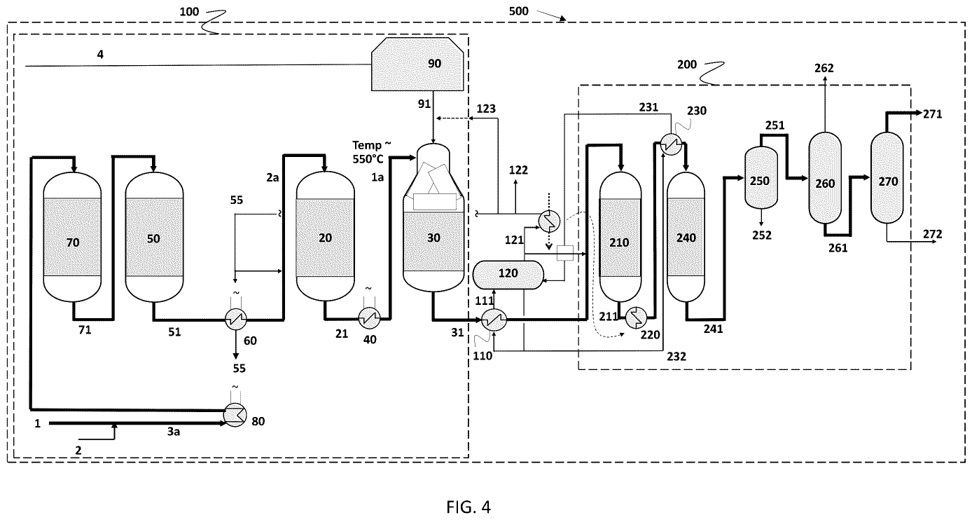

FIG. 4 shows a developed layout of a hydrogen plant comprising developed layout of the system of the invention.

### DETAILED DISCLOSURE OF THE INVENTION

Unless otherwise specified, any given percentages for gas content are % by volume. All feeds are preheated as required.

For the avoidance of doubt, the term “feed” refers to means for supplying said gas to the appropriate section, reactor or unit; such as a duct, tubing etc.

A “section” comprises one or more “units” which perform a change in the chemical composition of a feed, and may additionally comprise elements such as e.g. heat exchanger, mixer or compressor, which do not change the chemical composition of a feed or stream.

Similarly, a “stage” comprises one or more sections.

The term “synthesis gas” (abbreviated to “syngas”) is meant to denote a gas comprising hydrogen, carbon monoxide, carbon dioxide, steam and small amounts of other gasses, such as argon, nitrogen, methane, etc.

In a first aspects, a synthesis gas stage is provided. The output of such a stage is a synthesis gas (“syngas”) stream, e.g. comprising CO, H₂, H₂O, CO₂, CH₄ and mixtures thereof.

### Synthesis Gas Stage

In a first aspect, a synthesis gas stage is provided, said stage comprising:
a first hydrocarbon feed,
a prereformer section, arranged to receive the first hydrocarbon feed and provide a prereformed stream,
a reforming section, arranged to receive the prereformed stream and provide a first syngas stream,
wherein said synthesis gas stage comprises a first electrical heating unit located between said prereformer section and said reforming section, said first electrical heating unit being arranged to heat the prereformed stream to at least 400 °C, preferably at least 450 °C, prior to being fed to the reforming section.

A first hydrocarbon feed is provided. This first hydrocarbon feed suitably comprises a major portion of methane e.g. over 80%, such as over 90% of methane. Higher hydrocarbons (with >2 carbon atoms) may also be present. Suitably, the first hydrocarbon feed is a natural gas feed. The hydrocarbon feed may also comprise low amount of argon, nitrogen, carbon dioxide, steam and sulfides.

A prereformer section is arranged to receive the first hydrocarbon feed and provide a prereformed stream. Pre-reforming is the process by which methane and heavier hydrocarbons are steam reformed and the products of the heavier hydrocarbon reforming are methanated. A prereformer section may comprise an adiabatic pre-reformer filled with a catalyst with high nickel content and a main steam reformer. The adiabatic pre-reformer is usually positioned upstream of the main steam reformer. Steam may be added to the stream comprising hydrocarbons upstream the prereforming section. The provided prereformed stream comprises CO₂, CH₄, H₂O and H₂ along with typically lower quantities of CO and possible other components.

In the prereformers all higher hydrocarbons can be converted to carbon oxides and methane, but the prereformers are also advantageous for light hydrocarbons. Providing the prereformer may have several advantages including reducing the required O₂ consumption in the ATR and allowing higher inlet temperatures to the ATR since cracking risk by preheating is minimized. Furthermore, the prereformers may provide an efficient sulphur guard resulting in a practically sulphur free feed gas entering the ATR and the downstream system.

A reforming section is arranged to receive the prereformed stream (from the prereformer section) and provide a first syngas stream. Providing a prereformed stream to the reforming section may have several advantages including that the prereformer section may provide an efficient sulphur guard resulting in a practically sulphur free feed gas entering the reforming section and the downstream system.

The reforming section comprises at least one of an autothermal reforming (ATR) section, a reverse water gas shift (RWGS) section, optionally, where the reverse water gas shift section is electrically heated, a steam methane reformer (SMR) section, steam methane reformer-b (SMR-b) section, and/or a convection reformer (HTCR) section. In a preferred aspect, the reforming section comprises an autothermal reforming (ATR) section and the ATR section is arranged to receive a feed comprising an oxidant.

An ATR section may comprise one or more ATR reactors such that the key part of the ATR section is the one or more ATR reactor(s). An ATR reactor typically comprises a burner, a combustion chamber, and a catalyst bed contained within a refractory lined pressure shell. In an ATR reactor, partial combustion of hydrocarbons by sub-stoichiometric amounts of an oxidant such as oxygen is followed by steam reforming (reaction 1 and 2) of the partially combusted hydrocarbons in a fixed bed of steam reforming catalyst.

$$C {H}_{4} ( g ) + {H}_{2} O ( g ) ⇌ CO ( g ) + 3 {H}_{2} ( g ) \qquad ( 1 )$$

$${CH}_{4} ( g ) + 2 {H}_{2} O ( g ) ⇌ {CO}_{2} ( g ) + 4 {H}_{2} ( g ) \qquad ( 2 )$$

Steam reforming also takes place to some extent in the combustion chamber due to the high temperature. The steam reforming reaction is accompanied by the water gas shift reaction. Typically, the gas is at or close to equilibrium at the outlet of the reactor with respect to steam reforming and water gas shift reactions. More details of ATR and a full description can be found in the art such as “Studies in Surface Science and Catalysis, Vol. 152, “Synthesis gas production for FT synthesis”; Chapter 4, p. 258-352, 2004”.”.

As an ATR section requires sub-stoichiometric amounts of an oxidant such as oxygen for the partial combustion of hydrocarbons, a feed comprising an oxidant is provided to the ATR section. Suitably, the oxidant feed consists essentially of oxygen. The oxidant feed of O₂ is suitably “oxygen-rich” meaning that the major portion of this feed is O₂; i.e. over 75% such as over 90% or over 95%, such as over 99% of this feed is O₂. This oxidant feed may also comprise other components such as nitrogen, argon, CO₂, and/or steam. This oxidant feed will typically include a minor amount of steam (e.g. 5-10%). The source of oxidant feed, oxygen, can be at least one air separation unit (ASU) and/or at least one membrane unit. The source of oxygen can also be at least one electrolyser unit. A part or all of the oxidant feed of 02 may come from at least one electrolyser, which converts steam or water into hydrogen and oxygen by use of electrical energy. Steam may be added to the oxidant feed comprising oxygen, upstream the ATR section.

In a preferred aspect, the synthesis gas stage thus comprises a reforming section comprising an autothermal reforming (ATR) section and the ATR section is arranged to receive a feed comprising an oxidant.

In another preferred aspect, the synthesis gas stage comprises a reforming section comprising an autothermal reforming (ATR) section and further comprises an air separation unit and an air feed, said air separation unit being arranged to separate said air feed into at least an oxygen-rich stream and supply at least a portion of said oxygen-rich stream to the ATR section as said feed comprising an oxidant. Within this aspect, it should be noted that the amount of required O₂ consumption in the ATR reactor is reduced because the reforming section receives the prereformed stream said stream comprising mostly CO₂, CH₄, H₂O and H₂ compared to the alternative where the reforming section receives a non-prereformed hydrocarbon feed.

Typically, the effluent gas stream from the ATR reactor i.e. the first syngas stream has a temperature of 900-1100 °C. The syngas normally comprises hydrogen, carbon monoxide, carbon dioxide, and steam. Other components such as methane, nitrogen, and argon may also be present often in minor amounts. The operating pressure of the ATR reactor will be between 5 and 100 bars or more preferably between 15 and 60 bars.

The synthesis gas stage further comprises a first electrical heating unit located between said prereformer section and said reforming section. The first electrical heating unit is arranged to heat the prereformed stream to at least 400 °C, preferably at least 450 °C, prior to being fed to the reforming section. The presence of said prereformer section upstream said reforming section allows for higher inlet temperatures to the reforming section comprising at least one reactor such as at least one ATR reactor since cracking risk by preheating is minimized. Essentially said synthesis gas stage is arranged in such a way that there is no temperature change between the electrical heating unit and the reforming section.

In a conventional synthesis gas stage usually at least one fired heater is provided to preheat the prereformed stream upstream the inlet of the reforming section. To reduce carbon emission from the fired heater the inlet temperature of the reforming section is kept low such as below 450 °C such as 400 °C. To completely avoid the carbon emission the fired heater can be eliminated as well. However, removal of fired heater makes operation complex and makes it difficult to start-up. Hence, the synthesis gas stage is arranged to have an electric heater to heat the prereformed gas at the inlet of the reforming section such as at the inlet of an ATR section. Coupling the electrical heater with renewable power will also not impact the carbon emission from said synthesis gas stage or from a plant comprising said synthesis gas stage. In this way, an electrical heater with renewable power allows to provide green energy to enhance production such as H₂ production (aspect wherein said synthesis gas stage is comprised in a hydrogen production plant) for the given amount of O₂ without increasing the CO₂ emissions. In the preferred aspect, where the reforming section comprises an autothermal reforming (ATR) section and wherein the ATR section is arranged to receive a feed comprising an oxidant such as oxygen, the heating of the prereformed stream to at least 450 °C such as to 550 °C or more will reduce the oxygen consumption for the same syngas generation capacity or for the given oxygen flow the syngas generation capacity can be increased without any carbon emission.

In a conventional plant usually the fired heater is provided to preheat the prereformed stream inlet ATR. In order to reduce carbon emission from the fired heater the inlet temperature of ATR is kept low say below 450 °C such as 400 °C. To completely avoid the carbon emission the fired heater can be completely eliminated as well. However, removal of fired heater makes the plant operation little complex and also makes it difficult to start-up. Hence, the plant in the present invention is arranged to have an electric heater to heat the prereformed gas inlet ATR. Coupling the electrical heater with renewable power will also not impact the carbon emission from the plant. Heating the prereformed gas to at least 450 °C such 550 °C or more will reduce the oxygen consumption for the same syngas generation capacity or for the given oxygen flow the syngas generation capacity can be increased without any carbon emission.

Therefore, in a preferred aspect, the synthesis gas stage comprises a first electrical heating unit, wherein the first electrical heating unit is arranged to heat the prereformed stream to a temperature below 650 °C.

In other aspects, the reforming section within the synthesis gas stage can comprise a reverse water gas shift (RWGS) section, optionally, where the reverse water gas shift unit is electrically heated. Electrically heated reverse water gas shift (e-RWGS) uses an electric resistance-heated reactor to perform a more efficient reverse water gas shift process and substantially reduce or preferably avoids the use of fossil fuels as a heat source. The e-RWGS section comprise at least one e-RWGS reactor. In the e-RWGS reactor, either selective or non-selective RWGS may take place, wherein “selective RWGS” means that only the reverse water gas shift reaction in accordance with reaction 3,

$$C {O}_{2} ( g ) + {H}_{2} ( g ) ⇌ CO ( g ) + {H}_{2} O ( g ) . \qquad ( 3 )$$

takes place either on a catalyst or in a reactor while “non-selective RWGS” means that other reactions such as one or more of the methanation reactions (reverse of reaction 1 and 2) and reverse methanation takes place in addition to reverse water gas shift.

In a preferred aspect and as an example, the reforming section comprises a non-selective RWGS. The RWGS reaction (3) is an endothermic process which requires significant energy input for the desired conversion, however the simultaneous occurrence of methanation in the reactor results in release of chemical energy to heat the system and, thereby, a temperature increase as methanation reaction is an exothermic reaction. As the CO reduction reaction also is exothermic, the increase in temperature created by the methanation reaction results in a reduction of the potential for the CO reduction reaction and when the temperature has risen to a certain level no potential for the CO reduction reaction will be present at all. This exact level will be dependent on the specific reactant concentration, inlet temperature, and pressure, but will typically be in the range from 600-800 °C above which the CO reduction reaction will not have a potential to take place. Thus, said e-RWGS reactor allows increasing temperature over said reactor from relatively low inlet temperature such as 400-600 °C to a high product gas temperature in the reactor.

In this way, in a preferred aspect, the reforming section within the synthesis gas stage comprises an e-RWGS section and a first electrical heating unit located between said prereformer section and said reforming section, said first electrical heating unit being arranged to heat the prereformed stream to at least 400 °C, preferably at least 450 °C, prior to being fed to the reforming section.

In another aspect, the reforming section comprises a steam methane reforming (SMR) section or alternatively a SMR-b section, wherein the SMR-b section comprises a SMR section arranged in parallel to an e-RWGS section. In preferred aspects the SMR section is electrically heated such that it is a e-SMR section. Said e-SMR section comprises at least one SMR reactor wherein methane is heated with steam usually in the presence of a catalyst, to provide a mixture of carbon monoxide and hydrogen in accordance with reaction 1. An e-SMR reactor benefits from receiving a preheated feed such as a preheated prereformed hydrocarbon feed. In this way, a first electrical heating unit located between said prereformer section and said reforming section, said first electrical heating unit is, in a preferred aspect, arranged to heat the prereformed stream to at least 400 °C, preferably to at least 450 °C, prior to being fed to the reforming section.

In jet another aspect, the reforming section comprises a convection reforming section such as a Haldor Topsøe convection reforming (HTCR) section. Within this aspect, SMR and ATR reforming processes are integrated such that the conversion of hydrocarbons and steam to hydrogen and carbon oxides is fully autothermal, thereby avoiding any external fuel-fired heating. Thus said HTCR section comprises an integrated reactor designed in such a way that it comprises a primary reformer zone and a secondary reformer zone. Said primary reformer zone receives the prereformed stream and provide an SMR reformed effluent, which passes through a catalyst bed to the space at the feed end of the secondary reformer zone, at which preheated feed comprising an oxidant such as oxygen is introduced such that the secondary reformer zone provide a secondary reformer effluent. The hot secondary reformer effluent does not leave the reactor but passes on the shell side of the primary reformer zone, thereby applying the heat required for the endothermic SMR reforming reaction that occurs within the catalyst-containing reactor tubes of said primary reformer zone. Similarly, to the ATR, e-RWGS, and SMR reactors, this convection reforming comprising said integrated reactor benefits from receiving a preheated feed such as a preheated prereformed hydrocarbon feed and thereby by having a first electrical heating unit located between said prereformer section and said reforming section said first electrical heating unit is, in a preferred aspect, arranged to heat the prereformed stream to at least 400 °C, preferably to at least 450 °C, prior to being fed to the reforming section.

Also in a preferred aspect, the synthesis gas stage is composed such that said synthesis gas stage does not comprise a fired heater, in particular, wherein said section does not comprise a fired heater arranged to heat the prereformed stream prior to it being fed to the reforming section.

### Sulfur Removal Section

In one aspect, the synthesis gas stage further comprises a sulfur removal section arranged to receive a first hydrocarbon feed and provide a sulfur-depleted first hydrocarbon feed. Sulfur may be present as sulfides in the hydrocarbon feed, however it is not desirable to have sulfur in the stream entering the reforming section, as the presence of sulfur typically leads to contamination of catalysts such as carbon formation on the surface of said catalyst. Thus in a preferred aspect, the synthesis gas stage further comprises the sulfur removal section arranged upstream the reforming section and more preferred upstream the prereformer section.

Within the aspect where the synthesis gas stage comprises a sulfur removal section, the synthesis gas stage may further comprise a second electrical heating unit located between said sulfur removal section and said prereformer section. The second electrical heating unit is arranged to heat the sulfur-depleted first hydrocarbon feed to at least 400 °C, preferably at least 450 °C, prior to being fed to the prereformer section.

Thus, in a preferred aspect, the synthesis gas stage further comprises a sulfur removal section arranged to receive a first hydrocarbon feed, and provide a sulfur-depleted first hydrocarbon feed, said synthesis gas stage further comprises a second electrical heating unit, arranged to heat the sulfur-depleted first hydrocarbon feed, prior to said sulfur-depleted first hydrocarbon feed, being fed to the prereformer section.

### Hydrogenation Section

In one aspect, wherein the synthesis gas stage comprises a sulfur removal section, the synthesis gas stage may further comprise a hydrogenation section arranged to receive a first hydrocarbon feed and a hydrogen feed, optionally in admixture, and provide a hydrogenated first hydrocarbon feed to the sulfur removal section.

Within this aspect, the hydrogen feed is suitably “hydrogen rich” meaning that the major portion of this feed is hydrogen; i.e. over 75%, such as over 85%, preferably over 90%, more preferably over 95%, even more preferably over 99% of this feed is hydrogen. A part or all of the hydrogen feed may come from at least one electrolyser. An electrolyser means a unit for converting steam or water into hydrogen and oxygen by use of electrical energy. Also, within this aspect, the synthesis gas stage may further comprise a third electrical heating unit located before said hydrogenation section. The third electrical heating unit is arranged to heat the first hydrocarbon feed, or the mixture of first hydrocarbon feed and hydrogen feed, to at least 300 °C, preferably at least 350 °C, prior to being fed to the hydrogenation section.

In this way, a preferred aspect of the synthesis gas stage, further comprises a hydrogenation section arranged to receive a first hydrocarbon feed and a hydrogen feed, optionally in admixture, and provide a hydrogenated first hydrocarbon feed to the sulfur removal section, preferably wherein said synthesis gas stage further comprises a third electrical heating unit arranged to heat the first hydrocarbon feed, or the mixture of first hydrocarbon feed and hydrogen feed, prior to said first hydrocarbon feed, or the mixture of first hydrocarbon feed and hydrogen feed, being fed to the hydrogenation section.

### Process for Producing a First Syngas Stream

A process for producing a first syngas stream in the synthesis gas stage described is also provided, said process comprising the steps of:
a. feeding the first hydrocarbon feed to the prereformer section, and prereforming the first hydrocarbon feed to provide a prereformed stream,
b. feeding the prereformed stream to the first electrical heating unit, and heating the prereformed stream,
c. feeding the heated, prereformed stream to the reforming section, and subjecting it to reforming to provide a first syngas stream.

In one preferred aspect of the process, the prereformed stream is heated to a temperature of at least 400 °C, preferably at least 450 °C, in the first electrical heating unit.

In one preferred aspect of the process, the prereformed stream is heated to a temperature of below 650 °C.

In one preferred aspect of the process, the synthesis gas stage comprises an autothermal reforming (ATR) section and said process comprises the step of feeding said feed comprising an oxidant to the ATR section.

In one preferred aspect of the process, the synthesis gas stage comprises an autothermal reforming (ATR) section and further comprises an air separation unit and an air feed, said air separation unit being arranged to separate said air feed into at least an oxygen-rich stream and supply at least a portion of said oxygen-rich stream to the ATR section as said feed comprising an oxidant and said process comprises the step of: feeding the air feed to the air separation unit to provide at least an oxygen-rich stream and feeding a portion of said oxygen-rich stream to the ATR section.

### Plants

Provided is a hydrogen plant, an ammonia plant, a methanol plant and a synthetic fuel production plant comprising the described synthesis gas stage.

In a preferred aspect, a plant comprising the described synthesis gas stage is provided, wherein said synthesis gas stage comprises an ATR section. Within this preferred aspect, the plant is operated with a low steam-to-carbon ratio such as of 0.4 or such as of 0.6, where said steam-to-carbon ratio is the steam-to-carbon ratio on molar basis in the synthesis gas stage such as within the ATR section. The low steam-to-carbon ratio within the ATR section enables lower energy consumption and reduced equipment size as less steam/water is carried over in the plant.

In a preferred aspect, provided is also a plant where an ATR section is arranged for its pressure being lower than what normally would be expected for ATR section operation which typically is 30 bar g or higher, for instance 30-40 bar g such as 37.5 bar g. This enables the capture of even more carbon, e.g. 97% or more of the carbon in the hydrocarbon feed whilst at the same time not compromising the energy efficiency, in particular when combined with the steam-to-carbon ratio in the ATR section being 0.4 or 0.6 or higher such as 0.8.

### Hydrogen Plant

In a preferred aspect, a hydrogen plant comprising the synthesis gas stage described is provided, said hydrogen plant further comprising a shift section and a hydrogen purification section, wherein the shift section is arranged to receive the first syngas stream from the reforming section and provide a second syngas stream, and wherein the hydrogen purification section is arranged to receive the second syngas stream from the shift section and provide a hydrogen-rich stream and an off-gas stream.

In one aspect, the shift section comprises a high temperature (HT) shift unit and a low temperature shift unit, said high temperature shift unit being arranged to receive the first syngas stream from the reforming section and to provide a first shifted syngas stream. The first shifted syngas stream is subsequently fed to the low temperature (LT) shift unit for further shifting, thereby providing a second syngas stream, which is shifted according to the water gas shift (WGS) reaction (reaction 3).

In a preferred aspect, said high temperature (HT) shift unit may comprise a promoted zinc-aluminum oxide based high temperature shift catalyst. Within this aspect, when said plant is operated the steam-to-carbon ratio in the reforming and HT shift section are less than 2.6. The advantage of a low steam-to-carbon ratio within the reforming section and shift section is that it enables higher synthesis gas throughput compared to high steam-to-carbon ratio. Additionally, a low steam-to-carbon ratio requires smaller equipment in the front-end due to the lower total mass flow through the plant.

In preferred aspects, the temperature in the high temperature shift step is in the range 300-600 °C, such as 300-400 °C, such as 350-380 °C. In preferred aspect, the temperature in the low temperature shift step is in the range 180-300 °C, such as 200-250 °C.

In one aspect, the purification section is arranged to receive said second syngas stream and provide a hydrogen product stream and an off-gas stream.

In one aspect, the purification section comprises a separation unit in which process condensate comprising mostly water is removed from the product gas.

In one aspect, the purification section comprises a CO₂ removal section in which CO₂ is separated from the product gas, thereby providing a CO₂-rich stream and a CO₂-depleted stream. In this way, the CO₂ within the CO₂-rich stream can be captured and stored and/or used elsewhere. Including a CO₂ removal section in the hydrogen plant thus enables limited CO₂ emission from the plant.

In one aspect, the purification section comprises a pressure swing adsorption unit (PSA). Within this aspect, the pressure swing adsorption unit provide a hydrogen-rich stream i.e. a hydrogen product stream and an off-gas stream.

In a preferred aspect, the purification section comprises a separation unit arranged to receive said second syngas stream from the shift section, and to provide a process condensate and a first product gas stream, where the first product gas stream is sent to a CO₂ removal section in which CO₂ is separated such that the CO₂ removal section provides a CO₂-rich stream and a second CO₂-depleted product gas stream followed by the second CO₂-depleted product gas is arranged to be fed to the PSA unit such that the PSA unit is arranged to provide a hydrogen product stream and an off-gas stream.

Enabled by the provided synthesis gas stage comprising at least said first electrical heating unit, the hydrogen plant may be arranged to be operated such that CO₂ emission is further reduced. In general preheating of feeds reduces the complication of the plant layout and increases the carbon efficiency. Said preheating unit also helps in smooth and fast start-up of the plant. Thus, provided is also a method for reducing CO₂ emissions in a plant, said method comprising the step of heating a prereformed stream in the first electrical heating unit and heating the prereformed stream to a temperature of at least 400 °C, preferably at least 450 °C, in the first electrical heating unit.

To increase the carbon efficiency of the hydrogen plant, in one aspect, at least a portion of the off-gas stream provide by the hydrogen purification section may be arranged to be recycled to the synthesis gas stage such as to the inlet of the prereformer section or to the reforming section as feed gas. In a preferred aspect, the off-gas may be added to the prereformed stream upstream an ATR section.

### Ammonia Plant

In one aspect, an ammonia plant comprising the synthesis gas stage described is provided, said ammonia plant further comprising a purification section followed by an ammonia synthesis loop, and optionally a shift section is arranged up-steam said purification section. In this way, in a preferred aspect, a shift section is arranged to receive the first syngas stream from the reforming section and provide a shifted first syngas stream. The shifted first syngas stream is then fed as a feed to the purification section which is arranged to also receive a nitrogen-rich stream and to provide a process stream and an off-gas stream. The ammonia synthesis loop is then arranged to receive said process stream and to provide an ammonia product stream.

In a preferred aspect, the ammonia plant comprises the equivalent shift section and purification section as comprised in the hydrogen plant i.e. the ammonia plant comprises a shift section, a separation unit, a CO₂ removal section and a PSA unit. The specification given for said sections in the description of the hydrogen plant is therefore equally valid for said ammonia plant.

Thus, in a preferred aspect, the purification section comprises a separation unit arranged to receive said second syngas stream from the shift section, and to provide a process condensate and a first product gas stream, where the first product gas stream is sent to a CO₂ removal section in which CO₂ is separated such that the CO₂ removal section provides a CO₂-rich stream and a second CO₂-depleted stream followed by the second CO₂-depleted stream is arranged to be fed to the PSA unit such that the PSA unit is arranged to provide a hydrogen-rich stream and an off-gas stream. Within this aspect, the hydrogen-rich stream can be mixed with and nitrogen-rich stream in a preferred H₂/N₂ ratio of approximate 3 to provide a process stream to an ammonia synthesis loop.

In an alternative aspect, the the purification section of the ammonia plant may comprise one or more shift sections and CO₂ removal section followed by a molecular sieve dryer and a N₂ wash unit (NWU). After the shift sections and CO₂ removal section, the CO₂-depleted stream may contain residual CO and CO₂ together with small amounts of CH₄, Ar, He and H₂O. The CO₂ and H₂O are preferably removed before the NWU because they otherwise would freeze at the low operating temperature within the NWU. This may for example be done by adsorption in a molecular sieve dryer consisting of at least two vessels one in operation while the other is being regenerated. Nitrogen may be used as dry gas for re-generation. Provided by the N₂ wash unit is equivalent to earlier aspects said process steam.

The nitrogen for the NWU may be supplied by an air separation unit (ASU) which separates atmospheric air into at least a nitrogen-rich stream and an oxygen-rich stream. In a preferred aspect, the oxygen-rich stream is used in an ATR section and the nitrogen-rich stream in the NWU.

In the NWU, the hydrogen-rich stream is washed by liquid nitrogen in a column where CH₄, Ar, He and CO are removed. The purified syngas preferably contains only ppm levels of Ar and CH₄. After the NWU, a nitrogen-rich stream may be added to the stream in order to adjust the N₂ content to a preferred ratio H₂/N₂ ratio of 3 to provide a process stream to the ammonia synthesis loop. The ammonia synthesis loop is arranged to receive at least a portion of the process stream to provide an ammonia product stream.

In this way, the ammonia plant comprises a purification section and an ammonia synthesis loop, and optionally also comprising a shift section. The purification section is arranged to receive the first syngas stream from the reforming section to provide a process stream or, alternatively, wherein a shift section is arranged to receive the first syngas stream from the reforming section and provide a shifted first syngas stream. The shifted first syngas stream is feed to the purification section and the purification section is arranged to provide a process stream, and the ammonia synthesis loop is arranged to receive said process stream and to provide an ammonia product stream.

### Methanol Plant

A methanol plant comprising the described synthesis gas stage is also provided.

In one aspect, the methanol plant further comprises a methanol synthesis stage. This stage comprises a methanol synthesis section where the first syngas stream from the synthesis gas stage is first converted to a raw methanol stream followed by a methanol purification section where said raw methanol stream is purified to obtain a methanol product stream. The methanol synthesis stage generates a methanol purge gas stream, which typically contains hydrogen, carbon dioxide, carbon monoxide and methane. Additional components such as argon, nitrogen or oxygenates with two or more carbon atoms may also be present in smaller amount.

Thus, in a preferred aspect, a methanol plant comprising the synthesis gas stage described is also provided, said methanol plant further comprising a methanol synthesis section and a methanol purification section, wherein the methanol synthesis section is arranged to receive the first syngas stream from the reforming section and provide a raw methanol stream, and wherein the methanol purification section is arranged to receive the raw methanol stream from methanol synthesis section and provide a methanol product stream and an purge gas stream.

In one aspect, at least a portion of said methanol purge gas stream may be fed as an additional feed to the synthesis gas stage such as upstream an ATR section. In one aspect the purge gas may be added to the prereformed stream upstream the ATR section. In this way, the purge gas is recycled into the synthesis gas stage thereby increasing the over-all carbon efficiency of the plant. The methanol purge gas stream may be purified prior to feeding it to the synthesis gas section. Suitably, to avoid excessive build-up of inert components that may be present in the methanol purge gas, only a portion of said methanol purge gas stream may be fed to the synthesis gas section; and another portion of the methanol purge gas may be purged and/or used as fuel.

In particular when the plant comprises a methanol synthesis stage, the first syngas stream at the outlet of said synthesis gas stage has a module, as defined herein, in the range 1.80-2.30; preferably 1.90-2.20. The term “module” is defined as:

$$Module , M = \frac{( {H}_{2} - {CO}_{2} )}{( CO + {CO}_{2} )}$$

### Synthetic Fuel Production Plant

The invention also provides a synthetic fuel production plant comprising the synthesis gas stage described herein, said synthetic fuel production plant further comprising a synthetic fuel synthesis section e.g. comprising a Fischer-Tropsch synthesis section and a product purification section, wherein the synthetic fuel synthesis section is arranged to receive the first syngas stream from the reforming section and provide a raw product stream, and wherein the product purification section is arranged to receive the raw product stream from the synthetic fuel synthesis section and provide a product stream and an off-gas stream.

### Specific Embodiments of the Invention

FIG. 1 shows a first layout of the synthesis gas stage (100). A first hydrocarbon feed (1) such as a natural gas feed is fed to a prereformer section (20) which is arranged to provide a prereformed stream (21). The prereformed stream (21) is sent to a first electrical heating unit (40) in which the prereformed stream is heated to at least 400 °C, preferably at least 450 °C, such as 550 °C or more, thereby providing a heated first stream (1a). The heated first stream (1a) is then fed to a reforming section (30), where said reforming section (30) is arranged to provide a first syngas stream (31).

FIG. 2 shows a more developed layout of the synthesis gas stage (100), wherein the reforming section (30) comprises an oxidant-requiring section such as an autothermal reforming (ATR) section. Within this layout, the heated first stream (1a) and a feed (91) comprising an oxidant, preferably an oxygen-rich feed, are fed to a reforming section (30) comprising the ATR section. In the ATR section (30), the heated first stream (1a) and the oxygen-rich stream (91) are reacted to provide a first syngas stream (31).

FIG. 3 shows a further developed layout of the synthesis gas stage (100). Within this layout the first hydrocarbon feed (1) such as a natural gas feed is optionally mixed with a hydrogen feed (2). The first hydrocarbon feed (1) or the mixture is pre-heated to approx. 380 °C by a third electrical heating unit (80). The pre-heated stream (3a) is sent to the hydrogenation section (70) in which a hydrogenated first hydrocarbon feed (71) is formed and then sent to a sulfur removal section (50) to provide a sulfur-depleted first hydrocarbon feed (51). The sulfur-depleted first hydrocarbon feed (51) is heated to a temperature of approx. 450 °C in a second electrical heating unit (60) and mixed with a steam stream (55) to provide a heated second stream (2a). The heated second stream (2a) is fed to a prereformer section (20), which is arranged to provide a prereformed stream (21). The prereformed stream (21) is sent to a first electrical heating unit (40) in which the prereformed stream is heated to at least 400 °C, preferably at least 450 °C, such as at least 550 °C, thereby providing a heated first stream (1a). The heated first stream (1a) are then fed to a reforming section (30), preferably an oxidant-requiring section such as an ATR section.

In FIG. 3, the reforming section (30), is—in addition to the heated first stream (1a)—arranged to receive a feed (91) comprising an oxidant (preferably an oxygen-rich feed) which is provided by an air separation unit (90). Thus, the air separation unit (90) is arranged to receive an air feed (4) and arranged to separate said air feed (4) into at least an oxygen-rich stream (91) to supply at least a portion of said oxygen-rich stream (91) to the reforming section (30). In the reforming section (30), the heated first stream (1a) and the oxygen-rich stream (91) are reacted to provide a first syngas stream (31).

FIG. 4 shows a developed layout of a hydrogen plant (500) comprising synthesis gas stage (100) and synthesis stage (200). The description of said synthesis gas stage (100) laid out in the description of FIG. 3 also applies to FIG. 4. Thus, the synthesis gas stage (100) is arranged to provide a first synthesis gas stream (31) from the reforming section (30), wherein the reforming section (30) is preferably an ATR section. A first waste heat boiler (110) is arranged downstream the reforming unit (30) and provides an internal steam feed (111) for the steam drum (120).

Steam drum (120) is arranged to dry the steam by separating liquid water, and provide a first steam stream (121) as required. The steam drum is supplied with boiler feed water (not shown) corresponding to the steam production and boiler water purge (not shown).

A portion of the superheated steam stream is—in the illustrated embodiment—also to be mixed with the first syngas stream (31) downstream the first waste heat boiler. High temperature (HT) shift reactor (210) receives the first syngas stream (31) downstream the first waste heat boiler (110) and provides a first shifted syngas stream (211). Steam superheater (220) is arranged downstream the HT shift reactor, and superheats the first steam stream (121) from the steam drum (120) via heat exchange with the first shifted syngas stream (211) from the HT shift reactor (210).

Downstream the superheater (220), the shifted syngas stream is subsequently fed to the low temperature (LT) shift reactor (240) for further shifting. Second waste heat boiler (230) is located between HT and LT shift reactors, as shown to generate second internal steam feed (231) for the steam drum (120) via heat exchange of a boiler water stream from the steam drum (120) with the shifted syngas stream downstream steam superheater (220).

As shown in FIG. 4, steam stream (123) is mixed with oxygen-rich stream (91). After the LT shift, the product gas is passed to separator (250) in which process condensate 252 (comprising mostly water) is removed. Following this, the product gas (251) is passed to CO₂ removal section 260, where CO₂ is removed in the form of CO₂-rich stream (262). The product gas is passed to a pressure-swing adsorption (PSA) unit (270) for separation of hydrogen stream (271) and an off-gas stream (272), which can be exported as fuel.

### EXAMPLE

Examples 1~4 show calculations of various parameters, based on the layout in FIG. 4.

| Example | Units | 1 | 2 | 3 | 4 |
|---|---|---|---|---|---|
| Heater | | No | Electrical | Electrical | Fired |
| inlet ATR | | heater | heater | heater | heater |
| ATR inlet T (1a) | °C | 382 | 450 | 550 | 550 |
| Elec heater duty | MW | 0 | 5.69 | 14.38 | 0 |
| (inlet ATR) | | | | | |
| Heater inlet | | Electrical | Electrical | Electrical | Fired |
| Prereformer | | heater | heater | heater | heater |
| Prereformer inlet T | °C | 400 | 400 | 400 | 400 |
| (2a) | | | | | |
| Elec heater duty | MW | 2.25 | 2.23 | 2.22 | 0 |
| (inlet Prereformer) | | | | | |
| (2a) | | | | | |
| Heater inlet | | Electrical | Electrical | Electrical | Fired |
| Hydrogenator | | heater | heater | heater | heater |
| Hydrogenator inlet | °C | 380 | 380 | 380 | 380 |
| T (70) | | | | | |
| Elec heater duty | MW | 14.4 | 14.3 | 14.19 | 0 |
| (inlet Hydro- | | | | | |
| genator) (70) | | | | | |
| O2 consumption | Nm3/h | 50277 | 48937 | 46888 | 46888 |
| H2 production | Nm3/h | 200000 | 200000 | 200000 | 200000 |
| (271) | | | | | |
| NG feed flow (1) | Nm3/h | 85129 | 84644 | 83926 | 83926 |
| NG fuel flow | Nm3/h | 0 | 0 | 0 | 3458 |
| Carbon emission | Nm3/h | 0 | 0 | 0 | 3489 |
| from preheating | | | | | |

Example 1 has no electrical heater at the inlet of the ATR. Examples 2 and 3 differ in terms of the ATR inlet temperature. As can be seen, these three examples provide zero carbon emission from preheating. Example 4 is same as Example 3 but the electrical heaters are replaced with a fired heater. This example shows significant CO₂ emission from preheating.

The present invention has been described with reference to a number of aspects and figures. However, the skilled person is able to select and combine various aspects within the scope of the invention, which is defined by the appended claims. All documents referenced herein are incorporated by reference.

1. A synthesis gas stage, said stage comprising:
a first hydrocarbon feed,
a prereformer section, arranged to receive the first hydrocarbon feed and provide a prereformed stream,
a reforming section, arranged to receive the prereformed stream and provide a first syngas stream,
wherein said synthesis gas stage comprises a first electrical heating unit located between said prereformer section and said reforming section, said first electrical heating unit being arranged to heat the prereformed stream to at least 400 °C, preferably at least 450 °C, prior to being fed to the reforming section.

2. The synthesis gas section according to claim 1, wherein the first electrical heating unit is arranged to heat the prereformed stream to a temperature below 650 °C.

3. The synthesis gas stage according to claim 1, wherein said synthesis gas stage does not comprise a fired heater, in particular, wherein said stage does not comprise a fired heater arranged to heat the prereformed stream prior to it being fed to the reforming section.

4. The synthesis gas stage according to claim 1, further comprising a sulfur removal section arranged to receive a first hydrocarbon feed, and provide a sulfur-depleted first hydrocarbon feed, said synthesis gas stage further comprising a second electrical heating unit, arranged to heat the sulfur-depleted first hydrocarbon feed, prior to said sulfur-depleted first hydrocarbon feed, being fed to the prereformer section.

5. The synthesis gas stage according to claim 4, further comprising a hydrogenation section arranged to receive a first hydrocarbon feed and a hydrogen feed, optionally in admixture, and provide a hydrogenated first hydrocarbon feed to the sulfur removal section.

6. The synthesis gas stage according to claim 1, wherein the reforming section comprises at least one of an autothermal reforming section, a reverse water gas shift section, optionally, where the reverse water gas shift section is electrically heated, a steam methane reformer section, a steam methane reformer-b section, and/or a convection reformer section.

7. The synthesis gas stage according to claim 6, wherein the reforming section comprises an autothermal reforming section and wherein the ATR section is arranged to receive a feed comprising an oxidant.

8. The synthesis gas stage according to claim 7 further comprising an air separation unit and an air feed, said air separation unit being arranged to separate said air feed into at least an oxygen-rich stream and supply at least a portion of said oxygen-rich stream to the ATR section as said feed comprising an oxidant.

9. A process for producing a first syngas stream in the synthesis gas stage according to claim 1, said process comprising the steps of:
a. feeding the first hydrocarbon feed to the prereformer section, and prereforming the first hydrocarbon feed to provide a prereformed stream,
b. feeding the prereformed stream to the first electrical heating unit, and heating the prereformed stream,
c. feeding the heated, prereformed stream to the reforming section, and subjecting it to reforming to provide a first syngas stream.

10. The process according to claim 9, wherein the prereformed stream is heated to a temperature of at least 400 °C, in the first electrical heating unit.

11. The process according to claim 9, wherein the synthesis gas stage comprises an autothermal reforming section and where said process comprises the step of feeding said feed comprising an oxidant to the ATR section.

12. The process according to claim 9, wherein the synthesis gas stage comprises an autothermal reforming section and further comprises an air separation unit and an air feed, said air separation unit being arranged to separate said air feed into at least an oxygen-rich stream and supply at least a portion of said oxygen-rich stream to the ATR section as said feed comprising an oxidant and said process comprises the step of: feeding the air feed to the air separation unit to provide at least an oxygen-rich stream and feeding a portion of said oxygen-rich stream to the ATR section.

13. A hydrogen plant comprising the synthesis gas stage according to claim 1, said hydrogen plant further comprising a shift section and a hydrogen purification section, wherein the shift section is arranged to receive the first syngas stream from the reforming section and provide a second syngas stream, and wherein the hydrogen purification section is arranged to receive the second syngas stream from the shift section and provide a hydrogen-rich stream and an off-gas stream.

14. An ammonia plant comprising the synthesis gas stage according to claim 1, said ammonia plant further comprising a purification section and an ammonia synthesis loop, and optionally also comprising a shift section, and wherein the purification section is arranged to receive the first syngas stream from the reforming section to provide a process stream or, alternatively, wherein a shift section is arranged to receive the first syngas stream from the reforming section and provide a shifted first syngas stream, and wherein the shifted first syngas stream is feed to the purification section and the purification section is arranged to provide a process stream, and wherein the ammonia synthesis loop is arranged to receive said process stream and to provide an ammonia product stream.

15. A methanol plant comprising the synthesis gas stage according to claim 1, said methanol plant further comprising a methanol synthesis section and a methanol purification section, wherein the methanol synthesis section is arranged to receive the first syngas stream from the reforming section and provide a raw methanol stream, and wherein the methanol purification section is arranged to receive the raw methanol stream from the methanol synthesis section and provide a methanol product stream and an off-gas stream.

16. A synthetic fuel production plant comprising the synthesis gas stage according to claim 7, said synthetic fuel production plant further comprising a synthetic fuel synthesis section such as comprising a Fischer-Tropsch synthesis section and a product purification section, wherein the synthetic fuel synthesis section is arranged to receive the first syngas stream from the reforming section and provide a raw product stream, and wherein the product purification section is arranged to receive the raw product stream from the synthetic fuel synthesis section and provide a product stream and an off-gas stream.

17. A method for reducing CO₂ emissions in a hydrogen plant according to claim 13, said method comprising the step of heating a prereformed stream in the first electrical heating unit and heating the prereformed stream to a temperature of at least 400 °C, in the first electrical heating unit.
